(19) United States
(12) Patent Application Publication
(10) Pub. No.: US 2019/0233349 A1
(43) Pub. Date: Aug. 1, 2019

(54) ENHANCED SELECTIVITY TO C2+HYDROCARBONS BY ADDITION OF HYDROGEN IN FEED TO OXIDATIVE COUPLING OF METHANE

(71) Applicant: SABIC GLOBAL TECHNOLOGIES B.V., Bergen op Zoom (NL)

(72) Inventors: Sagar SARSANI, Sugar Land, TX (US); Aghaddin Khanlar MAMEDOV, Sugar Land, TX (US); David WEST, Sugar Land, TX (US); Wugeng LIANG, Sugar Land, TX (US)

(21) Appl. No.: 16/314,103

(22) PCT Filed: Jun. 23, 2017
(86) PCT No.: PCT/US2017/039073

§ 371 Date: Dec. 28, 2018

Related U.S. Application Data
(60) Provisional application No. 62/358,692, filed on Jul. 6, 2016.

(51) Int. Cl. C07C 2/84

(52) U.S. Cl. CPC C07C 2/84; C07C 2523/34

(57) ABSTRACT

A method of producing C2+ and higher hydrocarbons includes: (a) introducing a reactant mixture to a reactor having an oxidative coupling catalyst disposed therein, the reactant mixture including methane, oxygen, and hydrogen; (b) operating the reactor under such conditions that at least some of the methane of the reactant mixture undergoes an oxidative coupling reaction that gives rise to a product mixture that includes unreacted methane and primary products, the primary products including C2+ hydrocarbons; and (c) recovering at least a portion of the product mixture.

### TECHNICAL FIELD

The present disclosure relates to methods of producing hydrocarbons, and more particularly to producing olefins by oxidative coupling of methane.

### BACKGROUND

Hydrocarbons—specifically, olefins such as ethylene—are useful in a wide range of products, for example, break-resistant containers and packaging materials, among other things. For industrial scale applications, ethylene is produced by heating natural gas condensates and petroleum distillates, which include ethane and higher hydrocarbons, and the produced ethylene is separated from a product mixture by using gas separation processes.

Ethylene can also be produced by oxidative coupling of methane (OCM), as shown by Equations (1) and (2) below. Oxidative conversion of methane to ethylene is exothermic. Excess heat produced from these reactions (as shown in equations (1) and (2)) can push conversion of methane toward carbon monoxide and carbon dioxide rather than the desired C2 hydrocarbon product (e.g., ethylene). The excess heat from the reactions in equations (3) and (4) below further exacerbates, thereby substantially reducing the selectivity of ethylene production when compared with carbon monoxide and carbon dioxide production.

2CH₄+O₂═C₂H₄+2H₂O, ΔH=−67 kcal/mol (1)

2CH₄+½O₂═C₂H₄+H₂O, ΔH=−42 kcal/mol (2)

CH₄+1.5O₂═CO+2H₂O, ΔH=−124 kcal/mol (3)

CH₄+2O₂═CO₂+2H₂O, ΔH=−192 kcal/mol (4)

Although OCM is overall exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kilojoule per mole (kJ/mol)). When catalysts are used in OCM, the exothermic reaction can lead to a large increase in catalyst bed temperature and uncontrolled heat excursions that lead to catalyst deactivation and further decrease in ethylene selectivity. Furthermore, the produced ethylene is highly reactive and can form unwanted and thermodynamically favored oxidation products.

There have been attempts to control the exothermic reaction of the OCM by using alternating layers of selective OCM catalysts; through the use of fluidized bed reactors; and by using steam as a diluent. These solutions are, however, costly and inefficient. For example, a large amount of water (steam) is required to absorb the heat of the reaction.

These and other shortcomings are addressed by aspects of the present disclosure.

### SUMMARY

As described elsewhere—and without being bound by any particular theory—introducing hydrogen to a reactant mixture can generate active species (e.g., active radical species), for example by interaction with oxygen, which can further generate new routes for the OCM reaction in the absence of an OCM catalyst. Generally, a stoichiometric equation reaction of hydrogen with oxygen can be described by reaction (5):

2H₂+O₂=2H₂O (5)

Further, without wishing to be limited by theory, at high reaction temperatures (e.g., from about 700 degrees Celsius (° C.) to about 1,100° C.), hydrogen and oxygen can create hydroxyl radicals and can propagate an OCM reaction in the presence of methane according to reactions (6)-(9):

H₂+O₂=2OH. (6)

OH.+CH₄═H₂O+CH₃. (7)

CH₃.+O₂═CH₃O₂. (8)

CH₃O₂.═CH₂O+OH. (9)

Without wishing to be limited by theory, hydroxyl radical groups (e.g., OH.) as produced by reaction (6) can abstract hydrogen from methane as shown in reaction (7), which can generate radical active species (e.g., CH3.) for propagating the OCM reaction similarly to the generation of catalytic active species on a catalyst surface. Reaction (8) can significantly reduce C2 selectivity. Further, without wishing to be limited by theory, addition of hydrogen to the reactant mixture can (i) generate radicals by reaction (6) and (ii) consume oxygen, thereby decreasing the role of reaction (8).

In meeting the described challenges, the present disclosure provides methods of producing C2+ and higher hydrocarbons, comprising: (a) introducing a reactant mixture to a reactor having an oxidative coupling catalyst disposed therein, the reactant mixture comprising methane, oxygen, and hydrogen, (b) operating the reactor under such conditions that at least some of the methane of the reactant mixture undergoes an oxidative coupling reaction that gives rise to a product mixture that comprises unreacted methane and primary products, the primary products comprising C2+ hydrocarbons; and (c) recovering at least a portion of the product mixture.

The present disclosure also provides further methods, the further methods comprising: in a reactor that reacts methane and oxygen in the presence of an oxidative coupling catalyst so as to give rise to a product mixture that comprises C2+ hydrocarbons, introducing an amount of hydrogen to the reactor in an amount effective to increase a selectivity to C2+ hydrocarbons in the product mixture by from about 70 to about 99% relative to a corresponding reactor without hydrogen introduction.

Additionally provided are systems, comprising: a reactor having disposed therein an amount of an oxidative coupling catalyst, the reactor being configured to react methane, oxygen, and hydrogen in the presence of the oxidative coupling catalyst so as to give rise to a product mixture that comprises unreacted methane and primary products, the primary products comprising C2+ hydrocarbons; and a separation train configured to introduce at least a portion of the unreacted methane of the product mixture to the reactor.

### BRIEF DESCRIPTION OF THE DRAWINGS

The summary, as well as the following detailed description, is further understood when read in conjunction with the appended drawings. For the purpose of illustrating the invention, there are shown in the drawings exemplary and preferred aspects of the invention; however, the disclosure is not limited to the specific methods, compositions, and devices disclosed. In addition, the drawings are not necessarily drawn to scale. In the drawings:

FIG. 1 provides exemplary results from operating an OCM reactor with varying amounts of hydrogen added to a feed that comprises methane and oxygen.

### DETAILED DESCRIPTION OF ILLUSTRATIVE EMBODIMENTS

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

As described elsewhere, OCM has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically to form C₂H₄, water (H₂O) and heat. Generally, in the absence of an OCM catalyst, conversion of methane is low and the main products of conversion are CO and CO₂, as thermodynamically favored by reactions (3) and (4) shown above.

As disclosed here, catalytic OCM selectivity towards C2+ is enhanced when H₂ is added to the feed. Such a method can be particularly useful at commercial scale of operation, where H₂ is produced as a byproduct during the OCM reaction and does not have to be separated during recycling.

Without being bound to any particular theory, the positive effect of hydrogen is related to the tailoring of surface oxygen through removal of weekly-adsorbed oxygen by reaction with hydrogen. Removal of weakly-adsorbed oxygen species eliminates the reaction of non-selective conversion of methane to CO₂ with participation of that oxygen centers. Again without being bound to any particular theory, this approach leads to the increase of C2 selectivity, which is observed experimentally.

In this way, adding H₂ to the feed mixture to OCM increases selectivity. Such a method can be particularly useful at commercial scale of operation, where H₂ produced as a byproduct during the OCM reaction does not have to be separated during recycling. The amount of hydrogen may be adjusted to achieve a certain proportion to methane and oxygen. The optimal concentration of hydrogen added to a methane/oxygen mixture depends from the performance of the catalyst and the necessary amount of hydrogen may vary significantly depending on the Me-O bond of the catalyst.

For example, in the case of Na₂WO₄—Mn/SiO₂ catalyst, hydrogen in a methane/oxygen/hydrogen mixture may be from about 0-8%, relative to methane. In the presence of non-reducible catalysts such as Li/MgO, mixture of basic catalysts, CaO—La₂O₃, Sr—La₂O₃ the effect of hydrogen can be different from that observed in the case of Na₂WO₄—Mn/SiO₂ catalyst.

Selectivity

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈; C_{C4S}=number of moles of C from CH₄ that were converted into C₄ hydrocarbons (C_{4S}); C_{CO2}=number of moles of C from CH₄ that were converted into CO₂; C_{CO}=number of moles of C from CH₄ that were converted into CO, and so on.

The product mixture of the disclosed OCM processes may comprise coupling products, partial oxidation products (e.g., partial conversion products, such as CO, H₂, CO₂), and unreacted methane. In an aspect, the coupling products can comprise olefins (e.g., alkenes, characterized by a general formula CₙH₂ₙ) and paraffins (e.g., alkanes, characterized by a general formula CₙH₂ₙ₊₂).

The product mixture can comprise C₂₊ hydrocarbons and synthesis gas, wherein the C₂₊ hydrocarbons can comprise C₂ hydrocarbons and C₃ hydrocarbons. In an aspect, the C₂₊ hydrocarbons can further comprise C₄ hydrocarbons (C₄s), such as for example butane, iso-butane, n-butane, butylene, etc. A product mixture can comprise C₂H₄, C₂H₆, CH₄, CO, H₂, CO₂ and H₂O.

C₂ hydrocarbons can comprise ethylene (C₂H₄) and ethane (C₂H₆). In some aspects, a C₂H₄ content of the product mixture can be higher than a C₂H₆ content of the product mixture. In an aspect, the C₂ hydrocarbons can further comprise acetylene (C₂H₂). C₃ hydrocarbons can comprise propylene (C₃H₆). In an aspect, the C₃ hydrocarbons can further comprise propane (C₃H₈).

In some aspects, selectivity to primary products (e.g., Cₚₚ selectivity) can be from about 60% to about 99%, alternatively from about 70% to about 99%, alternatively from about 90% to about 99%, alternatively from about 75% to about 95%, or alternatively from about 80% to about 90%. The Cₚₚ selectivity refers to how much primary products (e.g., desired products, such as C₂ hydrocarbons, C₃ hydrocarbons, C₄s, CO for synthesis gas, etc.) were formed divided by the total products formed, including C₂H₄, C₃H₆, C₂H₆, C₃H₈, C₂H₂, C₄s, CO₂ and CO. For example, the Cₚₚ selectivity can be calculated by using equation (10):

$$C \text{?} selectivity = \frac{\begin{matrix}2 C \text{?} + 2 C \text{?} + 2 C \text{?} + 3 C \text{?} + \\ 3 C \text{?} + 4 C \text{?} + C \text{?}\end{matrix}}{\begin{matrix}2 C \text{?} + 2 C \text{?} + 2 C \text{?} + 3 C \text{?} + \\ 3 C \text{?} + 4 C \text{?} + C \text{?} + C \text{?}\end{matrix}} \times 100 \% \\ \text{?} \text{indicates text missing or illegible when filed} \qquad ( 10 )$$

As will be appreciated by one of skill in the art, if a specific product and/or hydrocarbon product is not produced in a certain OCM reaction/process, then the corresponding C_{Cx} is 0, and the term is simply removed from selectivity calculations.

In an aspect, a selectivity to ethylene (C₂=selectivity) can be from about 10% to about 60%, alternatively from about 15% to about 55%, alternatively from about 20% to about 50%, or alternatively from about 50% to about 65%. The C₂=selectivity refers to how much C₂H₄ was formed divided by the total products formed, including C₂H₄, C₃H₆, C₂H₆, C₃H₈, C₂H₂, C₄s, CO₂ and CO. For example, the selectivity to ethylene can be calculated by using equation (11):

$$C \text{?} selectivity = \frac{2 C \text{?}}{\begin{matrix}2 C \text{?} + 2 C \text{?} + 2 C \text{?} + 3 C \text{?} + \\ 3 C \text{?} + 4 C \text{?} + C \text{?} + C \text{?}\end{matrix}} \times 100 \% \\ \text{?} \text{indicates text missing or illegible when filed} \qquad ( 11 )$$

A selectivity to C₂ hydrocarbons (C₂ selectivity) can be from about 10% to about 70%, alternatively from about 15% to about 65%, or alternatively from about 20% to about 60%. The C₂ selectivity refers to how much C₂H₄, C₂H₆, and C₂H₂ were formed divided by the total products formed, including C₂H₄, C₃H₆, C₂H₆, C₃H₈, C₂H₂, C₄s, CO₂ and CO. For example, the C₂ selectivity can be calculated by using equation (12):

$$C \text{?} selectivity = \frac{2 C \text{?} + 2 C \text{?} + 2 C \text{?}}{\begin{matrix}2 C \text{?} + 2 C \text{?} + 2 C \text{?} + 3 C \text{?} + \\ 3 C \text{?} + 4 C \text{?} + C \text{?} + C \text{?}\end{matrix}} \times 100 \% \\ \text{?} \text{indicates text missing or illegible when filed} \qquad ( 12 )$$

A selectivity to C₂₊ hydrocarbons (C₂₊ selectivity) can be from about 15% to about 75%, alternatively from about 20% to about 70%, or alternatively from about 20% to about 65%. As described elsewhere herein, this selectivity to C₂₊ hydrocarbons may be at least about 70% or greater. The C₂₊ selectivity refers to how much C₂H₄, C₃H₆, C₂H₆, C₃H₈, C₂H₂, and C₄s were formed divided by the total products formed, including C₂H₄, C₃H₆, C₂H₆, C₃H₈, C₂H₂, C₄s, CO₂ and CO. For example, the C₂₊ selectivity can be calculated by using equation (13):

$$C \text{?} selectivity = \frac{\begin{matrix}2 C \text{?} + 2 C \text{?} + 2 C \text{?} + 3 C \text{?} + \\ 3 C \text{?} + 4 C \text{?}\end{matrix}}{\begin{matrix}2 C \text{?} + 2 C \text{?} + 2 C \text{?} + 3 C \text{?} + \\ 3 C \text{?} + 4 C \text{?} + C \text{?} + C \text{?}\end{matrix}} \times 100 \% \\ \text{?} \text{indicates text missing or illegible when filed} \qquad ( 13 )$$

Conversion

Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For example, methane conversion may be calculated by using equation (14):

$${CH}_{4} conversion = \frac{C \text{?} + C \text{?}}{C \text{?}} \times 100 \% \\ \text{?} \text{indicates text missing or illegible when filed} \qquad ( 14 )$$

Wherein Cⁱⁿ_{CH4} is the number of moles of C from CH₄ that entered the reactor as part of the reactant mixture, and C^{out}_{CH4} is the number of moles of C from CH₄ that was recovered from the reactor as part of the product mixture.

In an aspect, a sum of CH₄ conversion plus the selectivity to C₂₊ hydrocarbons can be equal to or greater than about 100%, alternatively equal to or greater than about 105%, or alternatively equal to or greater than about 110%. As will be appreciated by one of skill in the art, and with the help of this disclosure, the lower the residence time, the higher the selectivity to desired products, and the lower the methane conversion. Further, as will be appreciated by one of skill in the art, and with the help of this disclosure, the higher the reaction temperature, the higher the selectivity to desired products (e.g., olefins, hydrocarbons, etc.); however, generally, extremely high reaction temperatures (e.g., over about 1,100° C.) can lead to an increase in deep oxidation products (e.g., CO, CO₂).

In aspects where hydrogen is present in the reactant mixture, methane conversion and/or C₂₊ selectivity in an OCM reaction as disclosed herein can be increased when compared to a methane conversion and/or C₂₊ selectivity in an otherwise similar OCM reaction lacking H₂ in the reactant mixture. For example, methane conversion can be increased by equal to or greater than about 5%, alternatively equal to or greater than about 10%, or alternatively equal to or greater than about 15%, when compared to a methane conversion in an otherwise similar oxidative coupling of methane reaction conducted with a reactant mixture lacking hydrogen.

Further, selectivity to C₂₊ hydrocarbons can be increased by equal to or greater than about 5%, alternatively equal to or greater than about 10%, or alternatively equal to or greater than about 15%, when compared to a C₂₊ selectivity in an otherwise similar oxidative coupling of methane reaction conducted with a reactant mixture lacking hydrogen. For example, a selectivity to C₂₊ hydrocarbons may be increased from about 5% up to about 25%, or from about 10% to about 20%, or even by about 15%.

The disclosed methods can further effect minimizing deep oxidation of methane to CO₂. In an aspect, the product mixture can comprise less than about 15 mol % CO₂, alternatively less than about 10 mol % CO₂, or alternatively less than about 5 mol % CO₂.

In some suitable aspects, equal to or greater than about 2 mol %, alternatively equal to or greater than about 5 mol %, or alternatively equal to or greater than about 10 mol % of the reactant mixture can be converted to olefins. Equal to or greater than about 2 mol %, alternatively equal to or greater than about 5 mol %, or alternatively equal to or greater than about 10 mol % of the reactant mixture can be converted to ethylene. In some aspects, equal to or greater than about 4 mol %, alternatively equal to or greater than about 8 mol %, or alternatively equal to or greater than about 12 mol % of the reactant mixture can be converted to C₂ hydrocarbons.

In some aspects, equal to or greater than about 5 mol %, alternatively equal to or greater than about 10 mol %, or alternatively equal to or greater than about 15 mol % of the reactant mixture can be converted to C₂₊ hydrocarbons. In some aspects, equal to or greater than about 10 mol %, alternatively equal to or greater than about 15 mol %, or alternatively equal to or greater than about 20 mol % of the reactant mixture can be converted to synthesis gas. Generally, in industrial settings, synthesis gas is produced by an endothermic process of steam reforming of natural gas. In an aspect, the synthesis gas can be produced as disclosed herein as a side reaction in an OCM reaction/process.

Synthesis Gas and CO

A product mixture can comprise synthesis gas (e.g., CO and H₂). In an aspect, at least a portion of the H₂ found in the product mixture can be produced by the OCM reaction. Synthesis gas, also known as syngas, is generally a gas mixture consisting primarily of CO and H₂, and sometimes CO₂. Synthesis gas can be used for producing olefins; for producing methanol; for producing ammonia and fertilizers; in the steel industry; as a fuel source (e.g., for electricity generation); etc. In such aspect, the product mixture (e.g., the synthesis gas of the product mixture) can be characterized by a hydrogen (H₂) to carbon monoxide (CO) ratio of from about 0.5:1 to about 2:1, alternatively from about 0.7:1 to about 1.8:1, or alternatively from about 1:1 to about 1.75:1.

A selectivity to CO (C_{CO} selectivity) may, in some instances, be from about 25% to about 85%, alternatively from about 30% to about 82.5%, or alternatively from about 40% to about 80%. The C_{CO} selectivity refers to how much CO was formed divided by the total products formed, including C₂H₄, C₃H₆, C₂H₆, C₃H₈, C₂H₂, C₄s, CO₂ and CO. For example, C_{CO} selectivity can be calculated by using equation (15):

$${C}_{CO} selectivity = \frac{{C}_{CO}}{\begin{matrix}2 C \text{?} + 2 C \text{?} + 2 C \text{?} + 3 C \text{?} + \\ 3 C \text{?} + 4 C \text{?} + C \text{?} + C \text{?}\end{matrix}} \times 100 \% \\ \text{?} \text{indicates text missing or illegible when filed} \qquad ( 15 )$$

At least a portion of the synthesis gas can be separated from the product mixture to yield recovered synthesis gas, for example by cryogenic distillation. As will be appreciated by one of skill in the art, and with the help of this disclosure, the recovery of synthesis gas is done as a simultaneous recovery of both H₂ and CO. Similarly, at least a portion of the recovered synthesis gas can be further converted to olefins. For example, the recovered synthesis gas can be converted to alkanes by using a Fisher-Tropsch process, and the alkanes can be further converted by dehydrogenation into olefins.

Where desired, at least a portion of the unreacted methane and at least a portion of the synthesis gas can be separated from the product mixture to yield a recovered synthesis gas mixture, wherein the recovered synthesis gas mixture comprises CO, H₂, and CH₄. In an aspect, at least a portion of the recovered synthesis gas mixture can be further converted to olefins. In some aspects, at least a portion of the recovered synthesis gas mixture can be further used as fuel to generate power. In other aspects, at least a portion of the unreacted methane can be recovered and/or recycled to the reactant mixture. At least a portion of the recovered synthesis gas mixture can be further converted to liquid hydrocarbons (e.g., alkanes) by a Fisher-Tropsch process. In such aspects, the liquid hydrocarbons can be further converted by dehydrogenation into olefins.

At least a portion of the recovered synthesis gas mixture can be further converted to methane via a methanation process. The disclosed methods may comprise recovering at least a portion of the product mixture from the reactor, wherein the product mixture can be collected as an outlet gas mixture from the reactor. In an aspect, the product mixture can comprise primary products and unreacted methane, wherein the primary products comprise C₂₊ hydrocarbons and synthesis gas, and wherein the C₂₊ hydrocarbons comprise olefins. In an aspect, a method for producing olefins and synthesis gas can comprise recovering at least a portion of the olefins and/or at least a portion of the synthesis gas from the product mixture.

At least a portion of the C₂₊ hydrocarbons can be separated (e.g., recovered) from the product mixture to yield recovered C₂₊ hydrocarbons. The C₂₊ hydrocarbons can be separated from the product mixture by using any suitable separation technique. In an aspect, at least a portion of the C₂₊ hydrocarbons can be separated from the product mixture by distillation (e.g., cryogenic distillation). At least some of the recovered C₂₊ hydrocarbons can be used for ethylene production. In some aspects, at least a portion of ethylene can be separated from the recovered C₂₊ hydrocarbons to yield recovered ethylene, by using any suitable separation technique (e.g., distillation). In other aspects, at least a portion of the recovered C₂₊ hydrocarbons can be converted to ethylene, for example by a conventional steam cracking process.

In an aspect, at least a portion of the unreacted methane can be separated from the product mixture to yield recovered methane. Methane can be separated from the product mixture by using any suitable separation technique, such as for example distillation (e.g., cryogenic distillation). As described elsewhere herein, at least a portion of the recovered methane can be recycled to the reactant mixture.

Without being bound to any particular theory or condition, the disclosed methods for producing olefins and synthesis gas as disclosed herein at high temperatures (e.g., from about 700° C. to about 1,100° C.) and short residence times (e.g., from about 100 milliseconds to about 30 seconds) can advantageously provide for high C₂₊ selectivity along with synthesis gas with high H₂/CO molar ratio (e.g., up to about 2:1), wherein the selectivity to primary products can be very high (e.g., up to about 99%). Additional advantages of the methods for the production of olefins (e.g., ethylene) and synthesis gas as disclosed herein can be apparent to one of skill in the art viewing this disclosure.

Exemplary Aspects

Aspect 1. Methods of producing C₂₊ and higher hydrocarbons, comprising: (a) introducing a reactant mixture to a reactor having an oxidative coupling catalyst disposed therein, the reactant mixture comprising methane, oxygen, and hydrogen; (b) operating the reactor under such conditions that at least some of the methane of the reactant mixture undergoes an oxidative coupling reaction that gives rise to a product mixture that comprises unreacted methane and primary products, the primary products comprising C₂₊ hydrocarbons; and (c) recovering at least a portion of the product mixture.

A reactor may comprise an isothermal reactor, a fluidized sand bath reactor, an autothermal reactor, an adiabatic reactor, a tubular reactor, a cooled tubular reactor, a continuous flow reactor, a reactor lined with an inert refractory material, a glass lined reactor, a ceramic lined reactor, and the like, or combinations thereof. Inert refractory material can comprise silica, alumina, silicon carbide, boron nitride, titanium oxide, mullite, mixtures of oxides, and the like, or combinations thereof.

An isothermal reactor can comprise a tubular reactor, a cooled tubular reactor, a continuous flow reactor, and the like, or combinations thereof. An isothermal reactor can comprise a reactor vessel located inside a fluidized sand bath reactor, wherein the fluidized sand bath provides isothermal conditions (i.e., substantially constant temperature) for the reactor. In such aspects, the fluidized sand bath reactor can be a continuous flow reactor comprising an outer jacket comprising a fluidized sand bath. The fluidized sand bath can exchange heat with the reactor, thereby providing isothermal conditions for the reactor. Generally, a fluidized bath employs fluidization of a mass of finely divided inert particles (e.g., sand particles, metal oxide particles, aluminum oxide particles, metal oxides microspheres, quartz sand microspheres, aluminum oxide microspheres, silicon carbide microspheres) by means of an upward stream of gas, such as for example air, nitrogen, and the like.

A reactor can be a multi-stage reactor, wherein the multi-stage reactor can comprise multiple stages of reaction (e.g., OCM reaction). In an aspect, the multi-stage reactor can comprise from about 2 to about 10 reactors in series, alternatively from about 3 to about 8 reactors in series, or alternatively from about 4 to about 6 reactors in series. A multi-stage reactor can comprise any suitable number and arrangement of reactors (e.g., stages, reaction stages) in series and/or in parallel to achieve a desired methane conversion and selectivity to desired products. A selectivity to desired products obtained from a multi-stage reactor as disclosed herein may be higher than a selectivity to desired products obtained from a single stage reactor.

A multi-stage reactor can comprise one initial stage reactor, at least one intermediate stage reactor, and one finishing stage reactor. As will be appreciated by one of skill in the art, and with the help of this disclosure, the initial stage reactor, the intermediate stage reactor and the finishing stage reactor can each individually comprise any suitable number and arrangement of reactors (e.g., stages, reaction stages) in series and/or in parallel to achieve a desired methane conversion and selectivity to desired products.

An initial stage reactant mixture can be introduced to an initial stage reactor, wherein the initial stage reactant mixture can comprise methane, oxygen and optionally hydrogen. An intermediate stage reactant mixture can be introduced to an intermediate stage reactor, wherein the intermediate stage reactant mixture can comprise oxygen and optionally hydrogen. In an aspect, a finishing stage reactant mixture can be introduced to a finishing stage reactor, wherein the finishing stage reactant mixture can comprise oxygen. In an aspect, the initial stage reactor and the at least one intermediate stage reactor can operate at partial oxygen conversion, wherein the oxygen conversion can be from equal to or greater than about 50% to equal to or less than about 99%, alternatively from equal to or greater than about 55% to equal to or less than about 95%, or alternatively from equal to or greater than about 60% to equal to or less than about 90%. Near-complete oxygen conversion can be achieved in the finishing stage reactor, e.g., oxygen conversion in the finishing stage reactor can be equal to or greater than about 99%, alternatively equal to or greater than about 99.5%, or alternatively equal to or greater than about 99.9%.

Selectivity to C₂₊ hydrocarbons in a multi-stage reactor can be increased by equal to or greater than about 5%, alternatively by equal to or greater than about 10%, or alternatively by equal to or greater than about 15%, when compared to a selectivity to C₂₊ hydrocarbons of an otherwise similar oxidative coupling of methane reaction conducted in a single stage reactor.

The synthesis gas Hz/CO molar ratio produced by a multi-stage reactor as disclosed herein can be equal to or greater than about 1.0, alternatively equal to or greater than about 1.5, alternatively equal to or greater than about 1.9, or alternatively equal to or greater than about 2.

In an aspect, the synthesis gas Hz/CO molar ratio produced by a multi-stage reactor can be increased by equal to or greater than about 25%, alternatively equal to or greater than about 50%, or alternatively equal to or greater than about 100%, when compared to a synthesis gas Hz/CO molar ratio produced by an otherwise similar oxidative coupling of methane reaction conducted in a single stage reactor.

Isothermal conditions may be provided by fluidization of heated microspheres around the isothermal reactor comprising the catalyst bed, wherein the microspheres can be heated at a temperature of from about 675° C. to about 1,100° C., alternatively from about 700° C. to about 1,050° C., or alternatively from about 750° C. to about 1,000° C.; and wherein the microspheres can comprise sand, metal oxides, quartz sand, aluminum oxide, silicon carbide, and the like, or combinations thereof. In an aspect, the microspheres (e.g., inert particles) can have a size of from about 10 mesh to about 400 mesh, alternatively from about 30 mesh to about 200 mesh, or alternatively from about 80 mesh to about 100 mesh, based on U.S. Standard Sieve Size.

While in a fluidized state, individual inert particles become microscopically separated from each other by the upward moving stream of gas. Generally, a fluidized bath behaves remarkably like a liquid, exhibiting characteristics which are generally attributable to a liquid state (e.g., a fluidized bed can be agitated and bubbled; inert particles of less density will float while those with densities greater than the equivalent fluidized bed density will sink; heat transfer characteristics between the fluidized bed and a solid interface can have an efficiency approaching that of an agitated liquid; etc.).

Isothermal conditions can be provided by fluidized aluminum oxide, such as for example by a BFS high temperature furnace, which is a high temperature calibration bath, and which is commercially available from Techne Calibration.

While not required, a reaction mixture can be introduced to the reactor at a temperature of from about 150° C. to about 300° C., alternatively from about 175° C. to about 250° C., or alternatively from about 200° C. to about 225° C. As will be appreciated by one of skill in the art, and with the help of this disclosure, while the OCM reaction is exothermic, heat input may be useful in promoting the formation of methyl radicals from CH₄, as the C—H bonds of CH₄ are very stable, and the formation of methyl radicals from CH₄ is endothermic. The reaction mixture can be introduced to the reactor at a temperature effective to promote an OCM reaction.

A suitable reactant mixture can comprise a hydrocarbon or mixtures of hydrocarbons, and oxygen. In some aspects, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an aspect, the reactant mixture can comprise CH₄ and O₂.

Aspect 2. The method of aspect 1, wherein the reactor is maintained at a temperature (e.g., internal temperature) in the range of from about 750° C. to about 1000° C. For example, the reactor may be maintained at a temperature of from about 800° C. to about 900° C.

A diluent may be provided to the reactor, which diluent can provide for heat control of the OCM reaction, e.g., the diluent can act as a heat sink. Generally, an inert compound (e.g., a diluent) can absorb some of the heat produced in the exothermic OCM reaction, without degrading or participating in any reaction (OCM or other reaction), thereby providing for controlling a temperature inside the reactor. As will be appreciated by one of skill in the art, and with the help of his disclosure, a diluent can be introduced to the reactor at ambient temperature, or as part of the reaction mixture (at a reaction mixture temperature), and as such the temperature of the diluent entering the rector is much lower than the reaction temperature, and the diluent can act as a heat sink.

In some aspects, the product mixture can comprise C₂₊ hydrocarbons (including olefins), unreacted methane, synthesis gas and optionally a diluent. When water (e.g., steam) is used as a diluent, the water can be separated from the product mixture prior to separating any of the other product mixture components. For example, by cooling down the product mixture to a temperature where the water condenses (e.g., below 100° C. at ambient pressure), the water can be removed from the product mixture by using a flash chamber or other modality.

Aspect 3. The method of any of aspects 1-2, wherein at least a portion of the unreacted methane of the product mixture is introduced to the reactor. This may be accomplished by, e.g., a recycle line or lines that return at least some of the unreacted methane to the reactor. The unreacted methane may be separated from other products via various separation methods known to those of ordinary skill in the art. From 1 to about 100% of the unreacted methane may be introduced to the reactor, e.g., from 5 to 95%, from 10 to 90%, from 15 to 85%, from 20 to 80%, from 25 to 75%, from 30 to 70%, from 35 to 65%, from 40 to 60%, from 45 to 55%, or even 50% of the unreacted methane may be introduced to the reactor.

Aspect 4. The method of any of aspects 1-3, wherein the molar ratio of methane to oxygen introduced to the reactor may be from about 20:1 to about 2:1, e.g., from about 19:1 to about 2:1, from about 18:1 to about 2:1, from about 17:1 to about 2:1, from about 16:1 to about 2:1, from about 15:1 to about 2:1, from about 14:1 to about 2:1, from about 13:1 to about 2:1, from about 12:1 to about 2:1, from about 11:1 to about 2:1, from about 10:1 to about 2:1, from about 9:1 to about 2:1, from about 8:1 to about 2:1, from about 7:1 to about 2:1, from about 6:1 to about 2:1, from about 5:1 to about 2:1, from about 4:1 to about 2:1, or even from about 3:1 to about 2:1. A molar ratio of methane to oxygen introduced to the reactor of from about 8:1 to about 4:1 is considered especially suitable.

Aspect 5. The method of any of aspects 1-4, wherein the operating is substantially free of combustion. For example, the operating may be under conditions such that less than 50 mol %, less than 45 mol %, less than 40 mol %, less than 30 mol %, less than 35 mol %, less than 25 mol %, less than 20 mol %, less than 15 mol %, less than 10 mol %, less than 5 mol %, or even less than 1 mol % of the oxygen or methane provided to the reactor is combusted.

Aspect 6. The method of any of aspects 1-5, wherein the molar ratio of hydrogen to oxygen introduced to the reactor may be about 1:1 or less, e.g., 0.9:1, 0.8:1, 0.7:1, 0.6:1, 0.5:1, 0.4:1, 0.3:1, 0.2:1, or even about 0.1:1. A hydrogen to oxygen molar ratio of less than about 0.5:1 is considered particularly suitable.

The oxygen used in the reaction mixture can be oxygen gas (which may be obtained via a membrane separation process), technical oxygen (which may contain some air), air, oxygen enriched air, or combinations thereof.

Aspect 7. The method of any of aspects 1-6, wherein hydrogen introduced to the reactor is present at less than about 10 mol % relative to the total moles fed to the reactor. For example, hydrogen may be present at about 9 mol %, 8 mol %, 7 mol %, 6 mol %, 5 mol %, 4 mol %, 3 mol %, 2 mol %, or even 1 mol % relative to the total moles fed to the reactor.

Aspect 8. The method of any of aspects 1-7, wherein the reactor is operated such that at least about 10% of the methane introduced to the reactor is converted. For example, the reactor may be operated such that about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or even about 100% of the methane introduced to the reactor is converted.

Aspect 9. The method of any of aspects 1-8, wherein the reactor is operated such that at least about 80% of the oxygen introduced to the reactor is converted. For example, the reactor may be operated such that about 80%, about 85%, about 90%, about 95%, or even about 100% of the oxygen introduced to the reactor is converted.

Aspect 10. The method of any of aspects 1-9, wherein the reactor is operated such that a selectivity to primary products in the reactor is at least about 60%. For example, the selectivity may be about 60%, 65%, 70%, 75%, 80%, 85%, 90%, 95%, or even 99%. The selectivity may be from about 60% to about 85% (e.g., 83%), from 65% to about 80%, or even from about 70% to about 75%.

Aspect 11. The method of any of aspects 1-10, wherein the reactor is operated such that a selectivity to C2+ hydrocarbons is at least about 70%. For example, the selectivity may be about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or even 99%. A suitable selectivity range may be in the range of from about 77% to about 82%, including all intermediate values.

Aspect 12. The method of any of aspects 1-11, wherein the product mixture comprises hydrogen and wherein at least a portion of said hydrogen is introduced to the reactor. Hydrogen of the product mixture may be introduced to the reactor via one or more recycle lines. External hydrogen may also be introduced to the reactor; hydrogen fed to the reactor may comprise fresh hydrogen, recycled hydrogen, or both.

Aspect 13. The method of any of aspects 1-12, wherein the reactor is operated at a pressure of from about ambient pressure to about 500 pounds per square inch gauge (psig). For example, the reactor may be operated at from about ambient pressure to about 450 psig, 400 psig, 350 psig, 300 psig, 250 psig, 200 psig, 150 psig, or even from about ambient pressure to about 100 psig. The disclosed methods may be carried out at ambient pressure.

Aspect 14. The method of any of aspects 1-13, wherein the reactor is characterized by a gas hourly space velocity (GHSV) in the range of from about 50,000 to about 3,000,000 (hour)⁻¹ (h⁻¹). For example, the reactor may have a GHSV of from about 50,000 h⁻¹ to about 2,500,000 h⁻¹, or from about 100,000 h⁻¹, to about 2,000,000 h⁻¹, or from about 150,000 h⁻¹ to about 1,500,000 h⁻¹, or from about 200,000 h⁻¹ to about 1,000,000 h⁻¹, or even from about 250,000 h⁻¹ to about 500,000 h⁻¹. In some aspects, the reactor may have a GHSV of from about 30 h⁻¹ to about 20,000 h-1. GHSV may be measured at standard temperature and pressure.

Aspect 15. The method of any of aspects 1-14, further comprising recovering at least a portion of the primary products from the product mixture.

Aspect 16. The method of any of aspects 1-15, further comprising recovering ethylene from the primary products of the product mixture.

Aspect 17. A method, comprising: in a reactor that reacts methane and oxygen in the presence of an oxidative coupling catalyst so as to give rise to a product mixture that comprises C2+ hydrocarbons, introducing an amount of hydrogen to the reactor in an amount effective to increase a selectivity to C2+ hydrocarbons in the product mixture by from about 70% to about 99% relative to a corresponding reactor without hydrogen introduction.

As one example, a user may retrofit an existing OCM reactor system to include a feed of hydrogen; the hydrogen feed may in turn serve to improve the reactor's performance as described elsewhere herein. The hydrogen may be hydrogen that is evolved within the reactor, may be fresh hydrogen, or a combination of the two.

The selectivity increase may be from about 70% to about 99%, or from about 71% to about 98%, or from about 72% to about 97% or from about 73% to about 96% or from about 74% to about 95% or from about 75% to about 94% or from about 76% to about 93% or from about 77% to about 92% or from about 78% to about 91% or from about 79% to about 90% or from about 80% to about 89% or from about 81% to about 88% or from about 82% to about 87% or from about 83% to about 86% or from about 84% to about 85%.

Aspect 18. The method of aspect 17, wherein the hydrogen is introduced at a level of up to about 10 mol %. The level of hydrogen introduction is relative to the total moles introduced to the reactor. The hydrogen may be introduced at a level of about 10 mol %, about 9 mol %, about 8 mol %, about 7 mol %, about 6 mol %, about 5 mol %, about 4 mol %, about 3 mol %, about 2 mol %, or even about 1 mol %, relative to the total moles introduced to the reactor.

Aspect 19. The method of any of aspects 17-18, wherein (a) the ratio of methane to oxygen introduced to the reactor is from about 20:1 to about 2:1, (b) the ratio of hydrogen to oxygen introduced to the reactor is less than about 1:1, or both (a) and (b).

In some aspects, the molar ratio of methane to oxygen introduced to the reactor may be from about 20:1 to about 2:1, e.g., from about 19:1 to about 2:1, from about 18:1 to about 2:1, from about 17:1 to about 2:1, from about 16:1 to about 2:1, from about 15:1 to about 2:1, from about 14:1 to about 2:1, from about 13:1 to about 2:1, from about 12:1 to about 2:1, from about 11:1 to about 2:1, from about 10:1 to about 2:1, from about 9:1 to about 2:1, from about 8:1 to about 2:1, from about 7:1 to about 2:1, from about 6:1 to about 2:1, from about 5:1 to about 2:1, from about 4:1 to about 2:1, or even from about 3:1 to about 2:1. A molar ratio of methane to oxygen introduced to the reactor of from about 8:1 to about 4:1 is considered especially suitable.

In some aspects, the molar ratio of hydrogen to oxygen introduced to the reactor may be about 1:1 or less, e.g., 0.9:1, 0.8:1, 0.7:1, 0.6:1, 0.5:1, 0.4:1, 0.3:1, 0.2:1, or even about 0.1:1. A hydrogen to oxygen molar ratio of less than about 0.5:1 is considered particularly suitable.

Aspect 20. A system, comprising: a reactor having disposed therein an amount of an oxidative coupling catalyst, the reactor being configured to react methane, oxygen, and hydrogen in the presence of the oxidative coupling catalyst so as to give rise to a product mixture that comprises unreacted methane and primary products, the primary products comprising C₂₊ hydrocarbons; and a separation train configured to introduce at least a portion of the unreacted methane of the product mixture to the reactor. The separation train may also be configured to introduce at least a portion of any hydrogen in the product mixture to the reactor.

Suitable reactors are described elsewhere herein. Suitable OCM catalysts are known to those of ordinary skill in the art.

A separation train may comprise one, two, or more process units that are configured to introduce at least a portion of the unreacted methane of the product mixture to the reactor. Flash units, demethanizers, chillers, adsorption units, membrane separators, and the like may all be part of the separation train.

Systems according to the present disclosure may also comprise one or more units configured to separate one or more C₂₊ hydrocarbons (e.g., ethylene) from the product mixture, from the primary products, or both. Such units may include flash units, chillers, distillation units, adsorption units, membrane separators, and the like.

Aspect 21. The method of any of aspects 1-20, wherein the reactor comprises from about 2 to about 5 reactors. The reactors may be present in a series configuration, e.g., staged reactors. In such aspects, the first reactor may be considered an initial or first stage reactor.

Aspect 22. The method of aspect 21, wherein an initial stage reactant mixture comprising methane, oxygen and hydrogen is introduced to the initial stage reactor.

Aspect 23. The method of any of aspects 21-22, wherein at least a portion of the unreacted methane of the product mixture is introduced to the initial stage reactor.

Aspect 24. The method of any of aspects 21-23, wherein a reactor downstream from the initial stage reactor has introduced to it a product of a reactor upstream from the downstream reactor. The downstream reactor may also have introduced to it oxygen, hydrogen, or both. It should be understood that one or more of a series of reactors may have introduced therein hydrogen, oxygen, or both.

As one example, the third reactor in a series of five reactors may receive a product from the third of the series of five reactors. The third reactor may also have introduced therein hydrogen, oxygen, or both. The hydrogen and/or oxygen may be delivered from an external source, but may also be derived from one or more products of one or more reactors in the series.

### Illustrative Example

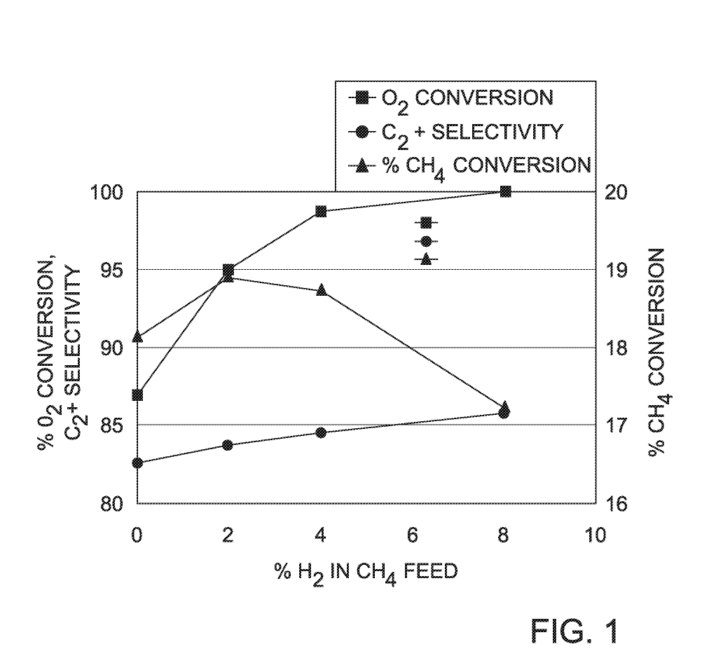

FIG. 1 provides illustrative, non-limiting results of operating OCM without and with varying amounts of H₂ added to the feed mixture at constant residence time through the catalyst bed containing Na₂WO₄—Mn—O/SiO₂ catalyst. When 2% H₂ (relative to CH₄) was added to the feed mixture, the conversion of oxygen and C2+ selectivity was increased, leading to enhanced methane conversion. (In the experiment exemplified in FIG. 1, the feed CH₄/O₂ ratio was 7.4, and the reactor temperature was 750° C. 100 milligrams (mg) of catalyst (Mn—Na₂WO₄/SiO₂) was used in a 4 millimeter (mm) inside diameter (I.D.) quartz reactor; the residence time was 54 milliseconds (ms)).

Another non-limiting example showing enhanced selectivity at a feed molar ratio of CH₄/O₂=4 at near complete O₂ conversion is presented in Table 1 below. The conditions for the experiment shown in Table 1 were: feed ratio of CH₄/O₂=4; reactor T=750° C.; 100 mg catalyst (Mn—Na₂WO₄/SiO₂); 4 mm I.D. quartz reactor, residence time=36 ms; near-complete O₂ conversion was observed.

TABLE 1

| H₂ % in CH₄ feed | 0 | 2 | 4 | 8 |
|---|---|---|---|---|
| CH₄ Conversion | 32.7 | 32.6 | 32.3 | 32.0 |
| C2+ Selectivity | 75.2 | 76.0 | 76.6 | 77.5 |

1. A method of producing C2+ and higher hydrocarbons, comprising:
(a) introducing a reactant mixture to a reactor having an oxidative coupling catalyst disposed therein,
the reactant mixture comprising methane, oxygen, and hydrogen;
(b) operating the reactor under such conditions that at least some of the methane of the reactant mixture undergoes an oxidative coupling reaction that gives rise to a product mixture that comprises unreacted methane and primary products,
the primary products comprising C2+ hydrocarbons; and
(c) recovering at least a portion of the product mixture.

2. The method of claim 1, wherein the reactor is maintained at a temperature in a range of from about 750° C. to about 1000° C.

3. The method of claim 1, wherein at least a portion of the unreacted methane of the product mixture is recycled to the reactor.

4. The method of claim 1, wherein a molar ratio of methane to oxygen introduced to the reactor is from about 20:1 to about 2:1.

5. The method of claim 1, wherein the operating is substantially free of combustion.

6. The method of claim 1, wherein a molar ratio of hydrogen to oxygen introduced to the reactor is less than about 1:1.

7. The method of claim 1, wherein the hydrogen introduced to the reactor is present at less than about 8 mol % in relation to total feed to the reactor.

8. The method of claim 1, wherein the reactor is operated such that at least about 10% of the methane introduced to the reactor is converted.

9. The method of claim 1, wherein the reactor is operated such that at least about 80% of the oxygen introduced to the reactor is converted.

10. The method of claim 1, wherein the reactor is operated such that a selectivity to primary products in the reactor is from about 60 to about 85%.

11. The method of claim 1, wherein the reactor is operated such that a selectivity to C2+ hydrocarbons is from about 75 to about 85%.

12. The method of claim 1, wherein the product mixture comprises hydrogen and wherein at least a portion of said hydrogen is introduced to the reactor.

13. The method of claim 1, wherein the reactor is operated at a pressure of from about ambient pressure to about 500 psig.

14. The method of claim 1, wherein the reactor is characterized by a gas hourly space velocity in a range of from 50,000 h⁻¹ to about 3,000,000 h⁻¹.

15. The method of claim 1, further comprising recovering at least a portion of the primary products from the product mixture.

16. The method of claim 1, further comprising recovering ethylene from the primary products of the product mixture.

17. A method, comprising:
in a reactor that reacts methane and oxygen in a presence of an oxidative coupling catalyst so as to give rise to a product mixture that comprises C2+ hydrocarbons,
introducing an amount of hydrogen to the reactor in an amount effective to increase a selectivity to C2+ hydrocarbons in the product mixture by from about 70 to about 99% relative to a corresponding reactor without hydrogen introduction.

18. The method of claim 17, wherein the hydrogen is introduced at a level of up to about 8 mol %.

19. The method of claim 17, wherein (a) a ratio of methane to oxygen introduced to the reactor is from about 20:1 to about 2:1, (b) a ratio of hydrogen to oxygen introduced to the reactor is less than about 1:1, or both (a) and (b).

20. A system, comprising:
a reactor having disposed therein an amount of an oxidative coupling catalyst,
the reactor being configured to react methane, oxygen, and hydrogen in a presence of the oxidative coupling catalyst so as to give rise to a product mixture that comprises unreacted methane and primary products,
the primary products comprising C₂₊ hydrocarbons; and
a separation train configured to introduce at least a portion of the unreacted methane of the product mixture to the reactor.
